(19) United States
(12) Patent Application Publication
(10) Pub. No.: US 2009/0118310 A1
(43) Pub. Date: May 7, 2009

(54) Activated Cdc42-associated kinase (ACK) as a therapeutic target for Ras-induced cancer

(75) Inventors: Alam Nur-E-Kamal, Edison, NJ (US); Ailing Zhang, Dayton, NJ (US); William J. Welsh, Princeton, NJ (US); Susan M. Keenan, Franklin Park, NJ (US); Xin I. Wang, Piscataway, NJ (US)

Correspondence Address: FOX ROTHSCHILD LLP;PRINCETON PIKE CORPORATE CENTER, 2000 Market Street, Tenth Floor, Philadelphia, PA, 19103, US

(73) Assignee: UNIVERSITY OF MEDICINE AND DENTISTRY OF NEW JERSEY, New Brunswick, NJ (US)

(21) Appl. No.: 11/463,535

(22) Filed: Aug. 9, 2006

Related U.S. Application Data
(60) Provisional application No. 60/706,655, filed on Aug. 9, 2005.

(51) Int. Cl. A61K 31/519; A61P 35/00

(52) U.S. Cl. 514/264.11

(57) ABSTRACT

Methods for preventing or treating Ras-induced cancer in a patient by (a) detecting v-Ha-Ras-transformed cells in a patient and (b) administering to the patient a therapeutically effective amount of a chemotherapeutic composition comprising an effective amount of an inhibitor for activated Cdc42-associated kinase (ACK) kinase.

### CROSS-REFERENCE TO RELATED APPLICATIONS

This application claims priority under 35 U.S.C. § 119(e) to U.S. Provisional Application Ser. No. 60/706,655 filed on Aug. 9, 2005, the disclosure of which is incorporated herein by reference.

### BACKGROUND OF THE INVENTION

Apoptosis is a mode of cell death in which the cell commits suicide either to ensure proper development of the organism or to destroy cells that represent a threat to the organism's integrity. There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J. Internal Medicine 237:529-536 (1995)). Pharmacological induction of apoptosis can be used to selectively destroy cancer-inducing cells.

v-Ha-Ras is an oncogenic mutant of Ras, which is a multieffector signaling molecule that has been implicated in the regulation of many cellular functions, including cell growth, differentiation, apoptosis, movement, and transformation (See Campbell et al., “Oncogenic Ras and its role in tumor cell invasion and metastasis,” Semin. Cancer Biol. 14:105-14 (2004); Lundberg et al., “Control of the cell cycle and apoptosis,” Eur. J. Cancer 35:1886-94 (1999)). Mutations in Ras genes that encode constitutively active proteins have been reported in at least 30% of human cancers (Macara et al., “The Ras superfamily of GTPases,” FASEB J. 10:625-30 (1996); McCormick et al., “Interactions between Ras proteins and their effectors,” Curr. Opin. Biotechnol. 7:449-56 (1996)); indeed, overexpression of Ras has been reported in various types of breast cancer and leukemia (Chang et al., “Regulation of cell cycle progression and apoptosis by the Ras/Raf/MEK/ERK pathway [review],” Int. J. Oncol. 22:469-80 (2003); Chang et al., “Involvement of PI3K/Akt pathway in cell cycle progression, apoptosis, and neoplastic transformation: a target for cancer chemotherapy,” Leukemia 17:590-603 (2003)). Furthermore, functional activation of a nononcogenic form of Ras contributes to the molecular pathogenesis of brain tumors and breast cancers (Bakin et al, “Constitutive activation of the Ras/mitogen-activated protein kinase signaling pathway promotes androgen hypersensitivity in LNCaP prostate cancer cells,” Cancer Res. 63:1981-9 (2003); Bakin et al., “Attenuation of Ras signaling restores androgen sensitivity to hormone-refractory C4-2 prostate cancer cells,” Cancer Res. 63:1975-80 (2003); Feldkamp et al., “Expression of activated epidermal growth factor receptors, Ras-guanosine triphosphate, and mitogen-activated protein kinase in human glioblastoma multiforme specimens,” Neurosurgery 45:1442-53 (1999)).

Therefore, a need exists for a method for selectively inducing apoptosis in oncogenic mutant Ras-transformed cells for treating Ras-associated disorders.

### SUMMARY OF THE INVENTION

This need is met by the present invention, which relates to a method of preventing or treating Ras-induced cancer in a patient by (a) detecting v-Ha-Ras-transformed cells in a patient and (b) administering to the patient a therapeutically effective amount of a chemotherapeutic composition comprising an effective amount of an inhibitor for activated Cdc42-associated kinase (ACK) kinase.

### BRIEF DESCRIPTION OF THE DRAWINGS

FIG. 1A demonstrates the involvement of Cdc42 in transducing Ras signals in inducing phosphorylation of ACK-1;

FIG. 1B demonstrates that Ras-Cdc42 signals for up-regulation of c-fos are transduced through ACK-1;

FIG. 1C demonstrates that the overexpression of the kinase mutant (K214R) of ACK-1 inhibits growth of v-Ras-transformed cells;

FIGS. 2A-D demonstrate the inhibition of v-Ha-Ras-transformed cell growth by ACK siRNA treatment;

FIGS. 3A-D show the induction of apoptosis by down-regulation of ACK in v-Ras-transformed NIH 3T3 cells;

FIGS. 4A-D demonstrate the inhibition of ACK kinase activity by kinase inhibitors;

FIGS. 5A-C demonstrate the inhibition of v-Ha-Ras-transformed cell growth by PD158780;

FIG. 6 is a visual representation of the three-dimensional structure of the kinase domain of ACK; FIG. 6A shows a-carbons depicted by a shaded ribbon with PD158780 in the binding pocket; FIG. 6B is an enlargement of ACK-PD158780 interaction; FIG. 6C is a structural drawing of PD158780; and

FIG. 7 depicts the ST021169 and ST038325 molecules and also demonstrates the growth inhibition effects of these compounds on v-Ha-Ras transformed cells.

### DETAILED DESCRIPTION OF THE INVENTION

The present invention derives from the discovery that down-regulation of activated Cdc42-associated kinase (ACK) induces apoptosis in v-Ha-Ras-transformed cells.

Ras is a multieffector signaling molecule that has been implicated in the regulation of many cellular functions, including cell growth, differentiation, apoptosis, movement, and transformation. v-Ha-Ras-transformed cells are NIH 3T3 cells, which express an oncogenic mutant of Ha-Ras protein and exhibit cancer cell phenotype. The small GTPase Cdc42 is involved in the transduction of Ras signals for the transformation of mammalian cells. Activated Cdc42-associated kinase (ACK) is an effector molecule for Cdc42.

The role of ACK in the transduction of Ras-Cdc42 signals for the survival of v-Ha-Ras-transformed cells has not been previously reported. Ras-Cdc42 signals transduced through ACK-1, an ACK isoform, protect v-Ha-Ras-transformed cells from apoptosis.

Therefore, the present invention relates to a method for preventing or treating Ras-induced cancer in a patient by (a) detecting v-Ha-Ras-transformed cells in a patient and (b) administering to the patient a therapeutically effective amount of a chemotherapeutic composition comprising an effective amount of an inhibitor for activated Cdc42-associated kinase (ACK) kinase. Preferred ACK inhibitors include PD158780, ST021169, and ST038325. Further, more than one ACK inhibitor can be included in the composition.

A cancer characterized by v-Ha-Ras-transformed cell growth in a patient can be treated by administering to the patient a therapeutically effective amount of a composition containing an ACK inhibitor. Treatable cancers include, but are not limited to breast cancer, pancreatic cancer, colon cancer, brain cancer, prostate cancer, and leukemia.

The composition can be administered to the patient prior to detecting Ras-induced cancer in the patient or after detecting Ras-induced cancer in the patient. The method can also include discontinuing the administration of the chemotherapeutic composition when v-Ha-Ras-transformed cells are no longer detectable in the patient. The ACK inhibitor may be administered alone or in combination with compounds known to be useful in the treatment of cancer.

In practice, a composition containing an inhibitor for ACK may be administered in any variety of suitable forms, some of which are related to tumor location, for example, by inhalation, topically, parenterally, rectally or orally; more preferably orally. More specific routes of administration include intravenous, intramuscular, subcutaneous, intraocular, intrasynovial, colonical, peritoneal, transepithelial including transdermal, ophthalmic, sublingual, buccal, dermal, ocular, nasal inhalation via insufflation, and aerosol.

A composition containing an inhibitor for ACK may be presented in forms permitting administration by the most suitable route. The invention also relates to administering pharmaceutical compositions containing at least one inhibitor for ACK which are suitable for use as a medicament in a patient. These compositions may be prepared according to the customary methods, using one or more pharmaceutically acceptable adjuvants or excipients. The adjuvants comprise, inter alia, diluents, sterile aqueous media and the various non-toxic organic solvents. The compositions may be presented in the form of oral dosage forms, or injectable solutions, or suspensions.

The choice of vehicle and the content of ACK inhibitor in the vehicle are generally determined in accordance with the solubility and chemical properties of the product, the particular mode of administration and the provisions to be observed in pharmaceutical practice. When aqueous suspensions are used they may contain emulsifying agents or agents which facilitate suspension. Diluents such as sucrose, ethanol, polyols such as polyethylene glycol, propylene glycol and glycerol, and chloroform or mixtures thereof may also be used. In addition, the ACK inhibitor may be incorporated into sustained-release preparations and formulations.

For parenteral administration, emulsions, suspensions or solutions of the compounds according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The injectable forms must be fluid to the extent that it can be easily syringed, and proper fluidity can be maintained, for example, by the use of a coating such as lecithin, by the maintenance of the required particle size in the case of dispersion and by the use of surfactants. Prolonged absorption of the injectable compositions can be brought about by use of agents delaying absorption, for example, aluminum monostearate and gelatin. The solutions of the salts of the products according to the invention are especially useful for administration by intramuscular or subcutaneous injection. Solutions of the ACK inhibitor as a free base or pharmacologically acceptable salt can be prepared in water suitably mixed with a surfactant such as hydroxypropyl-cellulose. Dispersion can also be prepared in glycerol, liquid polyethylene glycols, and mixtures thereof and in oils. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Sterile injectable solutions are prepared by incorporating the ACK inhibitor in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

Topical administration, gels (water or alcohol based), creams or ointments containing the ACK inhibitor may be used. The ACK inhibitor may be also incorporated in a gel or matrix base for application in a patch, which would allow a controlled release of compound through transdermal barrier.

For administration by inhalation, the ACK inhibitor may be dissolved or suspended in a suitable carrier for use in a nebulizer or a suspension or solution aerosol, or may be absorbed or adsorbed onto a suitable solid carrier for use in a dry powder inhaler.

The percentage of ACK inhibitor in the compositions used in the present invention may be varied, it being necessary that it should constitute a proportion such that a suitable dosage shall be obtained. Obviously, several unit dosage forms may be administered at about the same time. A dose employed may be determined by a physician or qualified medical professional, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 10, mg/kg body weight per day by intravenous administration. In each particular case, the doses are determined in accordance with the factors distinctive to the patient to be treated, such as age, weight, general state of health and other characteristics which can influence the efficacy of the compound according to the invention.

The ACK inhibitor used in the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the ACK inhibitor may be administered 1 to 4 times per day. Of course, for other patients, it will be necessary to prescribe not more than one or two doses per day.

The following non-limiting examples set forth hereinbelow illustrate certain aspects of the invention.

### EXAMPLES

### Materials

Cellfectin was purchased from Invitrogen Life Technologies (Carlsbad, Calif.). Isopropyl-L-thio-B-D-galactopyranoside, glutathione, MBP, DTT, and anti-phosphotyrosine were purchased from Sigma (St. Louis, Mo.). Glutathione-Sepharose was purchased from Amersham Biosciences (Uppsala, Sweden). FITC-VAD-fmk was purchased from Promega (Madison, Wis.). The polyclonal antibodies for c-fos and ACK-1 were purchased from Santa Cruz Biotechnology, Inc. (Santa Cruz, Calif.). [γ-³³P]ATP was purchased from NEN (Boston, Mass.). Ha-Ras antibodies and kinase inhibitors (PD158780, quercetin, wortmannin, PD157432, genistein, and radicicol) were purchased from Calbiochem (La Jolla, Calif.).

### General Procedures

Induction of c-fos Expression, Immunoprecipitation, And Western Blotting

NIH 3T3 cells (2.5×10⁵ per 35 mm dish) were cultured in DMEM supplemented with 10% FCS. After overnight incubation, cells were transfected with vector pMV7 (control), pMV7-ACKKR, pMV7-ACKLF, or v-Ras cDNA. Other cells were cotransfected with pMV7-ACKKR and v-Ras cDNA using the Cellfectin reagent. Each plasmid (2.5 μg) was mixed with 10 μg Cellfectin and left for 20 minutes to form complexes. The cells were then incubated with the DNA:Cellfectin complex for 2 hours in serum-free medium. The medium was replaced with medium containing 10% FCS for an additional 2 hours. The cells were then collected and lysed in Laemmli SDS sample buffer. v-Ha-Ras and Cdc42 mutants were transfected using the same protocol, except that cells were incubated overnight after transfection.

For immunoprecipitation, cells (2×10⁶ per sample) were lysed in a buffer containing 1% Triton X-100, 20 mmol/L HEPES (pH 7.4), 150 mmol/L NaCl, 10% glycerol, 1.5 mmol/L MgCl₂, 1 mmol/L EGTA, 10 mmol/L sodium pyrophosphate, 0.2 mmol/L sodium orthovanadate, 50 mmol/L NaF, 0.5 mg/mL phenylmethylsulfonyl fluoride, and 0.5 μg/mL aprotinin. Each lysate was incubated with ACK-1 antibody (10 μg/sample) for 4 hours at 4° C. Protein A-Sepharose CL-4B (50 μL) was added to the lysate followed by additional incubation for 2 hours at 4° C. Sepharose beads were collected by centrifugation at 1,000×g for 5 minutes (Eppendorf microfuge). The pellets were washed thrice with lysis buffer using the same protocol. Protein bound to Sepharose beads was recovered in Laemmli SDS sample buffer. Proteins were separated by SDS-PAGE and transferred to nylon membrane, and Western blotting was done according to the enhanced chemiluminescence protocol provided by the suppliers (Amersham Biosciences, Buckinghamshire, United Kingdom) using specific antibodies.

Plasmid Construction

A fragment of the ACK-1 gene (encoding amino acids 101-441) (SEQ ID NO: 1) corresponding to the SH3 and kinase domains (named ACKD) was amplified by oligonucleotide-directed PCR using primers (5′-GAATTCTTTGAGTACGTCAAGAATGAG-3′ and 5′-GAATTCTTAAAACGTGGGTCTGTCCTC-3′). The PCR product was digested with EcoRI and inserted into a bacterial expression vector, pGEX-2TH, using the EcoRI site. Accurate insertion of the PCR product was confirmed by nucleotide sequencing. Construction of the dominant-negative ACK mutant, ACK-1KR (K214R) is described in Kato et al., “Activation of the guanine nucleotide exchange factor Dbl following ACK1-dependent tyrosine phosphorylation.” Biochem. Biophys. Res. Comm. 268:141-7 (2000). The ACK-1 KR insert was digested with restriction endonuclease and transferred into the mammalian expression vector pMV-7.

Preparation of GST-ACK-1 Kinase Domain

Escherichia coli BL21 cells transformed with pGEX-ACKD were grown at 30° C. to early logarithmic phase and protein expression was induced by adding 0.1 mmol/L isopropyl-L-thio-β-D-galactopyranoside. After 3 hours of incubation, cells were harvested, resuspended in lysis buffer [50 mmol/L Tris (pH 7.5), 0.73 mol/L sucrose, 5 mmol/L MgCl₂, 0.5% (v/v) NP40], and disrupted by sonication. Cells were centrifuged at 10,000×g for 30 minutes at 4° C. The supernatant was applied to the glutathione-Sepharose column equilibrated with WED buffer [20 mmol/L Tris (pH 7.5), 2 mmol/L MgCl₂, 1 mmol/L DTT] followed by washing with WED buffer. GST-ACKD was eluted with 5 mmol/L glutathione solution in 50 mmol/L Tris (pH 9.6). The eluate was dialyzed in WED buffer overnight and concentrated on a sucrose gradient. The expected size of the fusion protein (GST-ACKD) was confirmed by SDS-PAGE (data not shown), and the protein was used for kinase assays as described below.

Kinase Assay

The purified GST-ACKD (˜5 μg per reaction) was incubated in kinase reaction buffer [50 mmol/L HEPES-KOH (pH 7.2), 10 mmol/L magnesium acetate, 5 mmol/L DTT] containing 7.5 μg MBP, 100 μmol/L ATP, and 4 μCi [γ-³³P]ATP for 10 minutes at 30° C. Reactions were stopped by addition of 5× Laemmli SDS sample buffer. Proteins were separated by SDS-PAGE, and radioactivity incorporated into the substrate was quantified by using the Kodak Imaging Station 2000R. For kinase inhibition experiments, GST-ACKD was preincubated with individual inhibitors in kinase buffer or kinase buffer alone (control) before the addition of MBP following the same protocol as described above. Experiments were done in triplicate.

Treatment of v-Ha-Ras-Transformed Cells With Kinase Inhibitors

To study the effects of ACK on cell proliferation, 2×10⁴ cells per well were seeded into 24-well plates and cultured under standard cell culture conditions. After overnight culture, individual kinase inhibitors (at indicated concentration) or DMSO (control) were added to the culture. Cells were collected by trypsinization after 48 hours. Cell numbers were counted with a hemocytometer. To study ACK phosphorylation, cells were lysed with a buffer containing 1% Triton X-100, 20 mmol/L HEPES (pH 7.4), 150 mmol/L NaCl, 10% glycerol, 1.5 mmol/L MgCl₂, 1 mmol/L EGTA, 10 mmol/L sodium pyrophosphate, 0.2 mmol/L sodium orthovanadate, 50 mmol/L NaF, 0.5 mg/mL phenylmethylsulfonyl fluoride, and 0.5 μg/mL aprotinin. Total protein (˜1 mg) was used for immunoprecipitation of ACK-1.

ACK siRNA Treatment

A pair of cRNA primers of 21 nucleotides (Dharmacon Research, Inc., Lafayette, Co.) corresponding to the 5′ noncoding of the ACK-1 cDNA (5′-CAUUACCCGCCUAUCUCAUdTdT-3′ and 5′-AUGAGAUAGGCGGGUAAUGdTdT-3′) were annealed to form siRNA (a 19-nucleotide duplex stem with two-nucleotide overhangs on either side) according to the instructions provided by the manufacturer. v-Ha-Ras-transformed or parental NIH 3T3 cells were seeded into 6- or 24-well plates and incubated overnight. The annealed double-stranded ACK siRNA (0.16, 0.4, or 0.8 nmol/L in DMEM) or the sense strand oligonucleotide of ACK siRNA (0.8 nmol/L) was complexed with Cellfectin. siRNA:Cellfectin complexes were added to the serum-free medium and incubated for 3 hours. Cells were then replenished with medium containing 10% FCS and incubated for another 21 hours or as indicated elsewhere. Cells were collected and counted using a hemocytometer; alternatively, cell lysates were prepared for Western blotting. Western blotting was done using ACK-1 antibodies.

Analysis of Cell Cycle Arrest And Induction of Apoptosis

v-Ras-transformed cells (1×10⁵) were seeded in a 35 mm dish and incubated under standard cell culture conditions overnight. Cells in DMEM were treated with Cellfectin, the sense strand of the siRNA:Cellfectin complex or the siRNA:Cellfectin complex for 3 hours. The medium was then replaced with DMEM containing 10% FCS and incubated for 21 hours at 37° C. Cells were harvested and used for Western blotting with specific antibodies or for cell cycle or caspase activation assays. For cell cycle and caspase activation assays, cells were resuspended in PBS containing FITC-VAD-fmk for 10 minutes at room temperature. The cells were then fixed with ice-cold 70% ethanol for 30 minutes at 4° C. Following a rinse with PBS, the cells were resuspended in PBS containing RNase (0.1 mg/mL) and then stained with propidium iodine (10 μg/mL) for 10 minutes at room temperature. Cellular fluorescence from a sample of 15,000 cells was analyzed using a Coulter EPICS Profile II Flow Cytometer (Coulter Electronics, Miami, Fla.). Fluorescence excited at 488 nm was detected using a 525±20 band pass filter. Histograms were analyzed using EPICS Workstation Software (version 4).

Nuclear DNA Fragmentation Assay

v-Ras-transformed cells (5×10⁵) were seeded in 35 mm dishes and incubated overnight under standard cell culture conditions. Cells in DMEM were treated for 3 hours with Cellfectin, Cellfectin complexed with the sense strand of siRNA, Cellfectin complexed with the siRNA, or VP-16. The medium was replaced with DMEM containing 10% FCS and cells were incubated for 21 hours at 37° C. Cells were harvested and chromosomal DNA fragmentation was assayed using methods described in Khelifa et al., “Induction of apoptosis by dexrazoxane (ICRF-187) through caspases in the absence of c-jun expression and c-Jun NH2-terminal kinase 1 (JNK1) activation in VM-26-resistant CEM cells,” Biochem. Pharmacol. 58:1247-57 (1999).

### Example 1

The involvement of ACK-1 in the transduction of Ras signals for transformation of mammalian cells was examined. NIH 3T3 cells were cultured in DMEM containing 10% FCS. Cells were transfected with vector alone, v-Ha-Ras, V12Cdc42, or v-Ha-Ras/N17Cdc42 constructs. Cells were lysed and ACK was immunoprecipitated as described above. Proteins obtained in the immunoprecipitate were separated by SDS-PAGE (8%), and Western blotting was done using antibodies against phosphotyrosine (P-Tyr). The amount of ACK-1 protein in each sample was determined by blotting the same membrane with antibodies against ACK-1.

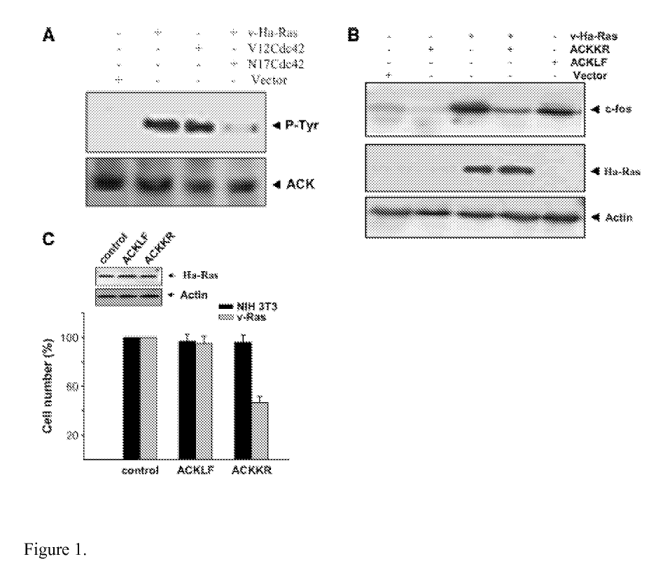

Expression of v-Ha-Ras in NIH 3T3 cells induces phosphorylation of ACK-1 (FIG. 1A), whereas coexpression of a dominant-negative mutant of Cdc42 blocked v-Ha-Ras-induced phosphorylation of ACK (FIG. 1A). This suggests that the Ras signal for ACK-1 phosphorylation is transduced through Cdc42.

The involvement of ACK-1 in transducing Ras signals for c-fos expression was then examined. A v-Ha-Ras (constitutively active) expressing plasmid was transfected into NIH 3T3 cells. NIH 3T3 cells were cultured as described above. Cells were transfected with pMV7 (vector) as control, pMV7-ACKKR, pMV7-ACKLF, v-Ras cDNA or were cotransfected with pMV7-ACKKR and v-Ras cDNA using Cellfectin reagent. After 4 hours, cells were collected and lysed in Laemmli SDS sample buffer. Proteins were separated by SDS-PAGE (10%), and Western blotting was done using c-fos or Ha-Ras antibodies. Equal loading of total protein was confirmed by blotting the membrane with actin antibodies.

Expression of v-Ha-Ras upregulated c-fos, whereas transfection of vector alone had no effect on c-fos levels (FIG. 1B). Cotransfection of a kinasedead mutant (K214R) of ACK-1 with the v-Ras construct into NIH 3T3 cells inhibited v-Ras-induced up-regulation of c-fos (FIG. 1B).

The kinase mutant (K214R) of ACK-1 was then expressed in v-Ha-Ras-transformed and parental NIH 3T3 cells. Normal and v-Ha-Ras-transformed NIH 3T3 cells were transfected with Cellfectin alone or were complexed with pMV7 (control), pMV7-ACKLF, or pMV7-ACKKR. After 4 hours, the transfection reagent-containing medium was replaced with DMEM containing 10% FCS, and cells were incubated under standard cell culture conditions. After 48 hours, cells were collected by trypsinization and their number was counted using a hemocytometer and compared with the number obtained for a vector alone transfection sample. In a parallel experiment, cell lysates were separated by SDS-PAGE (10%). Western blotting was done using antibodies against Ha-Ras. Equal loading of protein was confirmed by blotting the membrane with anti-actin antibodies.

K214R significantly inhibited the growth of v-Ha-Ras transformed cells. The expression of the K214R had no effect on the expression of c-fos, or on the growth of normal NIH 3T3 cells (FIGS. 1B and C), despite similar levels of K214R expression in transformed and parental NIH 3T3 cells (data not shown). Although the constitutively active mutant of ACK-1 (L543F) induced c-fos expression in NIH 3T3 cells (FIG. 1B), the L543F mutant, with similar levels of expression in each cell type (data not shown), had no effect on the growth of parental and v-Ha-Ras-transformed NIH 3T3 cells (FIG. 1C). Transfection of K214R and L543F did not alter the level of Ras expression in v-Ha-Ras-transformed cells (FIG. 1C), suggesting that inhibition of cell proliferation was not due to loss of Ras expression. These results indicate that ACK-1 is involved in transducing Ras signals and that ACK-1-dependent signals play a critical role in growth of v-Ha-Ras-transformed mammalian cells.

### Example 2

To further investigate whether ACK-1 is required for growth and survival of v-Ha-Ras-transformed cells, the expression of ACK-1 was knocked down using siRNA. v-Ha-Ras-transformed NIH 3T3 (FIGS. A and C) and parental NIH 3T3 (FIGS. B and D) cells were cultured in DMEM containing 10% FCS. Cells were treated with Cellfectin, Cellfectin complexed with the sense strand of ACK-1 siRNA, or ACK-1 siRNA.

After 24 hours of transfection, cells were collected and lysed with Laemmli SDS sample buffer. Proteins were separated by SDS-PAGE and the level of ACK-1 protein was determined by Western blotting using antibodies against ACK-1. Equal loading of total proteins was confirmed by blotting the membrane with actin antibodies.

After transfection with siRNA at different concentrations (in nmol/L), v-Ha-Ras transformed and parental NIH 3T3 cells were trypsinized and collected every 24 hours. Cell numbers were counted in triplicate.

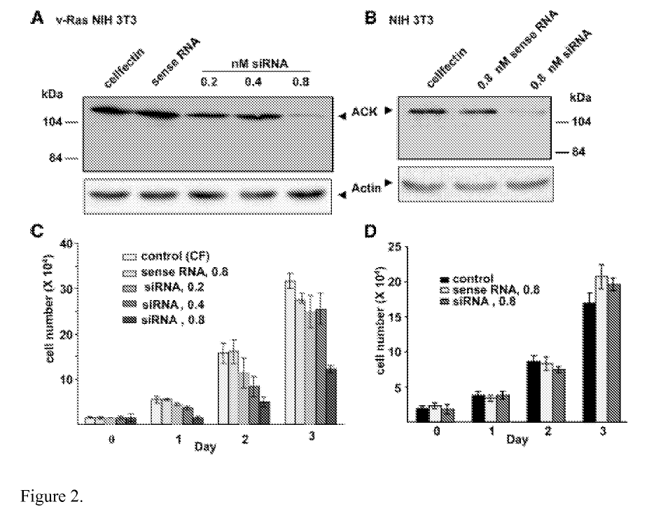

Transfection of ACK-1 siRNA reduced the expression of ACK-1 in a dose-dependent manner; 0.8 nmol/L ACK siRNA reduced the level of ACK-1 significantly in v-Ha-Ras-transformed and parental NIH 3T3 cells (FIGS. 2A and B). Transfection of ACK-1 siRNA similarly inhibited the growth of v-Ha-Ras-transformed cells in a dose dependent manner (FIG. 2C), whereas transfection of sense strand of siRNA did not affect the growth of v-Ha-Ras transformed NIH 3T3 cells (FIG. 2C). However, transfection of ACK-1 siRNA did not affect the growth of parental NIH 3T3 cells (FIG. 2D). Therefore, v-Ha-Ras-transformed cells, but not normal cells, may be dependent on ACK-1-mediated growth and fail to produce sufficient survival signals when the ACK-1-dependent Ras signaling pathway is interrupted. These results suggest an important involvement of ACK-1 in controlling the growth and survival of v-Ha-Ras-transformed mammalian cells.

A stable NIH 3T3 cell line was developed, which overexpressed either wild-type ACK-1 or a constitutively activated kinase mutant (L543F) of ACK-1. Neither ACK-1 nor the L543F mutant of ACK-1 produced a transformation phenotype in the transformation assay (data not shown). These results indicate that ACK alone is not sufficient to induce transformation of NIH 3T3 cells.

### Example 3

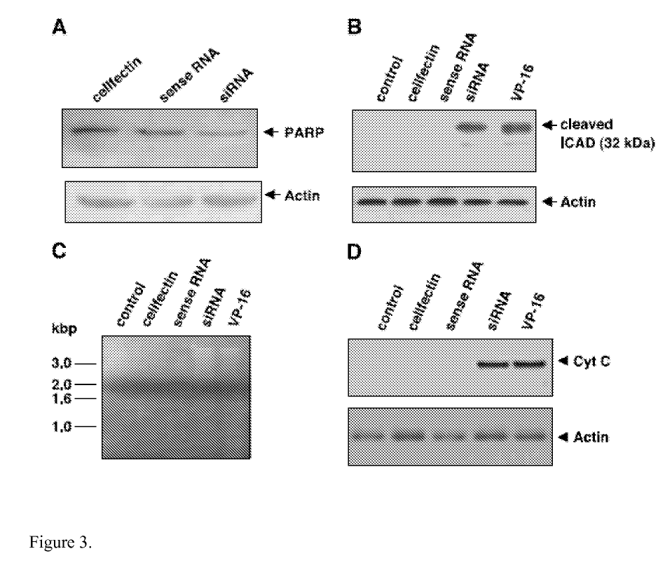

To investigate whether ACK-1 deficiency induces apoptosis in v-Ha-Ras-transformed cells, ACK siRNA was transfected into v-Ha-Ras-transformed cells to knockdown the expression of ACK-1. v-Ha-Ras-transformed NIH 3T3 cells were treated with DMEM (control), Cellfectin, Cellfectin complexed with the sense strand of ACK-1 siRNA, or ACK-1 siRNA. Treatment of cells with DNA topoisomerase II inhibitor, etoposide (VP-16), was done to provide a positive control. After 24 hours, cells were collected. Cells were lysed to get the total cellular proteins or fractionated to get cytoplasmic proteins as described in Nur-E-Kamal et al., “Nuclear translocation of cytochrome c during apoptosis,” J. Biol. Chem. 279:24911-4 (2004). Proteins (total cellular and cytoplasmic) were separated by SDS-PAGE, and Western blotting was done using antibodies against poly(ADP-ribose) polymerase (PARP) (FIG. 3A), inhibitor of caspase-activated DNase (ICAD) (FIG. 3B), and cytochrome c (Cyt C) (FIG. 3D). Equal loading of total protein was confirmed by blotting the membrane with antibodies against actin.

Cells were treated with Cellfectin, Cellfectin complexed with the sense strand of siRNA, or siRNA. Cells were collected after 21 hours. An equal number of untreated (control) and VP-16-treated cells were also collected after 21 hours of incubation. The cytoplasmic fraction was isolated, and DNA fragments were extracted and purified by ethanol precipitation. Isolated DNA fragments were characterized by 1.5% agarose gel electrophoresis. The experiment was repeated thrice showing similar results.

We found that transfection of ACK-1 siRNA induced apoptosis as determined by studying apoptosis markers, such as poly(ADP-ribose) polymerase cleavage (FIG. 3A), cleavage of the inhibitor of caspase-activated DNase (FIG. 3B), release of cytochrome c from mitochondria (FIG. 3D), and fragmentation of chromosomal DNA (FIG. 3C). Transfection of ACK siRNA did not block v-Ha-Ras-transformed cells at any particular stage of the cell cycle (data not shown), suggesting that ACK deficiency induced cell death in a cell cycle-independent manner. Collectively, these results suggest that Ras signals transduced through ACK-1 are required to protect v-Ha-Ras-transformed cells from apoptosis.

### Example 4

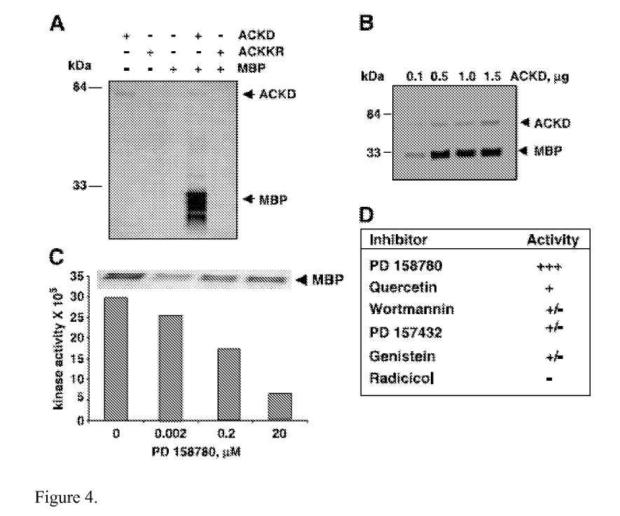

Several compounds were screened to examine their potency in inhibiting the kinase activity of ACK in vitro. The polypeptide (ACKD), which corresponds to the kinase and SH3 domains of ACK-1 (amino acids 101-441), was cloned in a bacterial expression vector, produced as a glutathione S-transferase (GST)-fusion protein (GST-ACKD), and affinity purified. A fragment of ACK-1 kinase (ACKD) and its K214R kinase mutant (ACKKR) were produced in E. coli and affinity purified as GST-fusion proteins. Kinase activity of the bacterially produced GST-fusion proteins was assayed using MBP as a substrate. Reaction products were characterized by SDS-PAGE followed by autoradiography (FIG. 4A). ACKD phosphorylated MBP in a dose-dependent manner (FIG. 4B).

Different kinase inhibitors were added to the ACK-1 kinase reaction as described above in General Procedures. Phosphorylation of MBP was determined by SDS-PAGE and autoradiography. The level of MBP phosphorylation was determined by scanning MBP bands using Kodak Imaging Station 2000R and plotted as arbitrary units. PD158780 inhibited ACK strongly and in a dose-dependent manner (FIG. 4C). The effect of independent kinase inhibitors at a concentration of 200 nmol/L is shown in FIG. 4D. Each of these experiments was repeated thrice showing similar results.

GST-ACKD exhibited autokinase activity as well as phosphorylated myelin basic protein (MBP) (FIGS. 4A and B). PD158780 and PD157432 were studied for their ability to inhibit the kinase activity of GST-ACKD in vitro. PD158780 has the strongest inhibitory activity, whereas quercetin, genistein, wortmannin, and PD157432 exhibited very weak activity (FIGS. 4C and D).

### Example 5

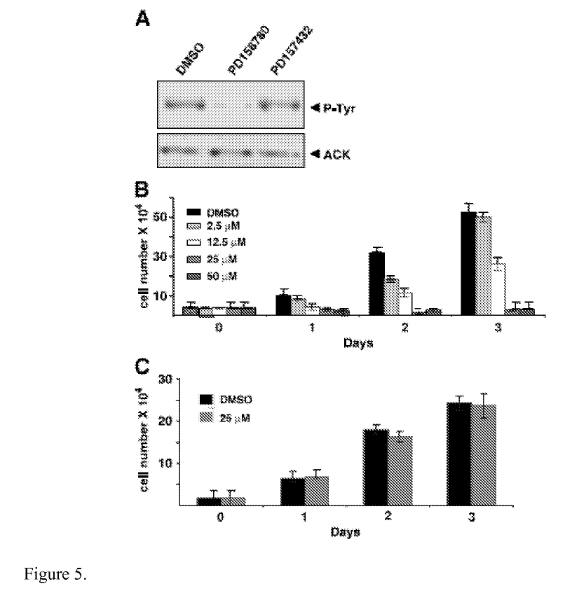

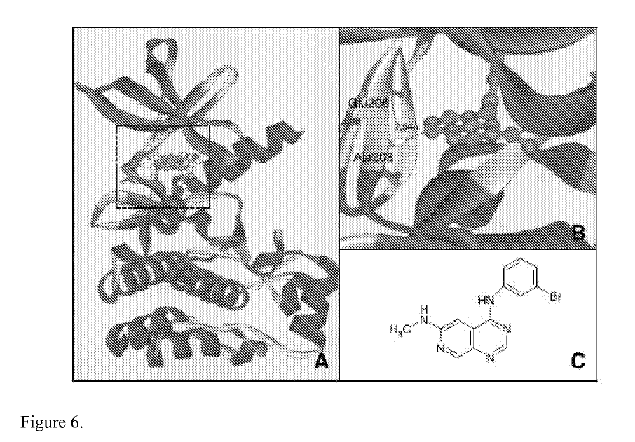

The effect of PD158780 and PD157432 on the phosphorylation of ACK-1 was investigated. v-Ha-Ras-transformed cells were cultured in DMEM containing 10% FCS. Cells were treated with solvent (DMSO), PD 158780 (25 μmol/L), or PD157432 (25 μmol/L) for 48 hours. Cells were incubated under standard cell culture conditions. For ACK-1 immunoprecipitation, cells were lysed and ACK-1 was immunoprecipitated as described above in General Procedures. Proteins present in the immunoprecipitate were separated by SDS-PAGE (8%), and Western blotting was done using antiphosphotyrosine antibody (FIG. 5A). Equal loading of ACK-1 was confirmed by blotting the same membrane with antibodies against ACK-1. For growth inhibition studies, cells were trypsinized and counted every 24 hours. The growth of v-Ras-transformed cells in the presence or absence of PD158780 (FIG. 5B) and PD157432 (FIG. 5C) were plotted.

It was found that PD158780 inhibited ACK-1 autophosphorylation to a much stronger extent than did PD157432 (FIG. 5A). These results suggest that PD158780 inhibits ACK kinase in v-Ras-transformed cells. Whether incubation with PD158780 or PD157432 affected the growth of v-Ha-Ras-transformed NIH 3T3 cells was then examined. After treatment with PD158780, v-Ha-Ras-transformed NIH 3T3 cell growth was inhibited in a dose-dependent manner (FIG. 5A), whereas PD157432 did not show any inhibitory effect (FIG. 5B). The differential abilities of the inhibitors to modulate ACK-1 phosphorylation and activity correlate strongly with their effects on the growth of v-Ha-Ras-transformed cells (FIG. 5).

### Example 6

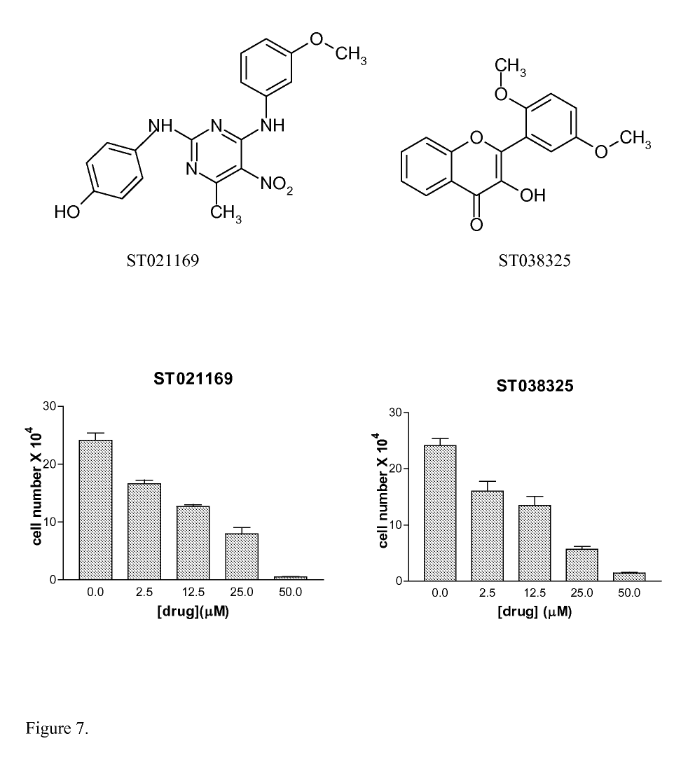

The effect of ST021169 and ST038325 on v-Ha-Ras-transformed NIH 3T3 cell growth was investigated. 2×10⁴ v-Ha-Ras-transformed cells per well were seeded into 24 well plates and cultured under standard cell culture conditions. After overnight culture, ST021169 or ST038325, at indicated concentrations, or DMSO (control) were added to the culture. After 48 hours, the cells were trypsinized and counted with a hemocytometer. The data indicates that incubation with ST021169 and ST038325 affected the growth of v-Ha-Ras transformed cells in a dose-dependent manner. (FIG. 7).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

What is claimed is:

1. A method for preventing or treating Ras-induced cancer in a patient comprising: (a) detecting v-Ha-Ras-transformed cells in a patient and (b) administering to said patient a therapeutically effective amount of a chemotherapeutic composition comprising an effective amount of an inhibitor for activated Cdc42-associated kinase (ACK) kinase.

2. The method of claim 1, wherein the inhibitor is selected from the group consisting of PD158780, ST021169, and ST038325.

3. The method of claim 1 comprising administering said composition prior to detecting Ras-induced cancer in said patient or after detecting Ras-induced cancer in said patient.

4. The method of claim 1, wherein said Ras-induced cancer comprises breast cancer, brain cancer, prostate cancer, pancreatic cancer, colon cancer, or leukemia.

5. The method of claim 1 further comprising discontinuing the administration of said chemotherapeutic composition when v-Ha-Ras-transformed cells are no longer detectable in said patient.

6. The method of claim 1 wherein said composition further comprises a pharmaceutically acceptable carrier.


SEQUENCE LISTING (5 sequences)

SEQ ID NO: 1 - protein, 341 aa, Artificial
  ACK-1 fragment
FEYVKNEDLEKIGMGRPGQRRLWEAVKRRKALCKRKSWMSKVFSGKRLEAEFPPHHSQSTFRKTSPAPGGPAGEGPLQSLTCLIGEKDLRLLEKLGDGSFGVVRRGEWDAPSGKTVSVAVKCLKPDVLSQPEAMDDFIREVNAMHSLDHRNLIRLYGVVLTPPMKMVTELAPLGSLLDRLRKHQGHFLLGTLSRYAVQVAEGMGYLESKRFIGRDLAARNLLLATRDLVKIGDFGLMRALPQNDDHYVMQEHRKVPFAWCAPESLKTRTFSHASDTWMFGVTLWEMFTYGQEPWIGLNGSQILHKIDKEGERLPRPEDCPQDIYNVMVQCWAHKPEDRPTF

SEQ ID NO: 2 - DNA, 27 bp, Artificial
  oligonucleotide primer
gaattctttgagtacgtcaagaatgag

SEQ ID NO: 3 - DNA, 27 bp, Artificial
  oligonucleotide primer
gaattcttaaaacgtgggtctgtcctc

SEQ ID NO: 4 - DNA, 21 bp, Artificial
  cRNA primer
cauuacccgccuaucucautt

SEQ ID NO: 5 - DNA, 21 bp, Artificial
  cRNA primer
augagauaggcggguaaugtt